(19) United States
(12) Patent Application Publication
(10) Pub. No.: US 2017/0368207 A1
(43) Pub. Date: Dec. 28, 2017

(54) SELF-ASSEMBLED NANOSTRUCTURE AND METHOD FOR PREPARING THE SAME

(71) Applicant: National Chiao Tung University, Hsinchu City (TW)

(72) Inventors: Ting-Yu Kuo, Changhua County (TW); Wei-Ting Huang, Taitung County (TW); Dean-Mo Liu, Hsinchu County (TW)

(73) Assignee: National Chiao Tung University, Hsinchu City (TW)

(21) Appl. No.: 15/298,256

(22) Filed: Oct. 20, 2016

(30) Foreign Application Priority Data
Jun. 24, 2016 (TW) 105119828

(51) Int. Cl. A61K 49/18; A61K 49/00

(52) U.S. Cl. CPC A61K 49/1818; A61K 49/0093; A61K 49/1824

(57) ABSTRACT

A self-assembled nanostructure including an amphiphilic chitosan and a contrast agent compound is provided. The contrast agent compound is grafted to the amphiphilic chitosan. The chemical bonding between the amphiphilic chitosan and the contrast agent compound has a synergistic effect to further improve the contrasting ability of the contrast agent compound.

### CROSS-REFERENCE TO RELATED APPLICATION

This application claims the priority benefit of Taiwan application serial no. 105119828, filed on Jun. 24, 2016. The entirety of the above-mentioned patent application is hereby incorporated by reference herein and made a part of this specification.

### BACKGROUND OF THE INVENTION

### Field of the Invention

The invention relates to a nanostructure and a method for preparing the same, particularly to a self-assembled nanostructure having contrasting ability and a method for preparing the same.

### Description of Related Art

Magnetic resonance imaging (MRI), because of its non-invasiveness and non-radiation, is an important tool for diagnosing a disease and identifying the stage of development of the disease. In MRI, contrast agents are usually utilized for improving resolution of a tissue image.

In the prior art, a metal-containing compound, such as a superparamagnetic iron oxide or a derivative thereof, is often used as a contrast agent. However, such iron oxide is likely to accumulate in tissue of an organism due to its longer half-life, and the issue about the occurrence of chronic toxicity over time is raised.

### SUMMARY OF THE INVENTION

In view of the above, the invention provides a self-assembled nanostructure and a method for preparing the same, and the prepared self-assembled nanostructure has excellent contrasting ability and has no biological toxicity.

A self-assembled nanostructure including an amphiphilic chitosan and a contrast agent compound is provided. The contrast agent compound is grafted to the amphiphilic chitosan.

In an embodiment of the invention, the amphiphilic chitosan includes carboxymethyl hexanoyl chitosan (CHC), deoxycholic acid modified carboxymethylated chitosan (DCMC), lauroyl sulfated chitosan (LSC) or methylpyrrolidone chitosan (MPC).

In an embodiment of the invention, the contrast agent compound includes a magnetic resonance imaging (MRI) contrast agent compound.

In an embodiment of the invention, the MRI contrast agent compound includes gadodiamide, gadopentetate dimeglumine, gadoterate meglumine or a combination thereof.

In an embodiment of the invention, the amphiphilic chitosan has a weight average molecular weight of about 1,000 to 60,000.

In an embodiment of the invention, the self-assembled nanostructure is entangled to form a spherical nanoparticle having a particle size of about 5 nm to 500 nm.

In an embodiment of the invention, the contrast agent compound is in an amount of about 1 part by weight to 30 parts by weight based on 100 parts by weight of the self-assembled nanostructure.

In an embodiment of the invention, the self-assembled nanostructure encapsulates a fluorescence contrast agent compound.

In an embodiment of the invention, the fluorescence contrast agent compound includes fluorescein isothiocyanate (FITC), C₈₃H₉₅N₁₃O₂₃S₂Zn₂ or C₅₇H₅₈N₁₄O₁₈SZn₂.

In an embodiment of the invention, the self-assembled nanostructure encapsulates a drug molecule.

In an embodiment of the invention, the drug molecule includes cis-diamminedichloridoplatinum(II) or a derivative thereof, gemcitabine or a derivative thereof; or demethoxycurcumin or a derivative thereof.

The invention provides a method for preparing a self-assembled nanostructure, including the following steps. An amphiphilic chitosan-containing solution is prepared. A contrast agent compound is added to the solution. A cross-linking agent is added to the solution. A cross-linking reaction is caused between the amphiphilic chitosan and the contrast agent compound. The solution is purified. The solution is dried.

In an embodiment of the invention, the cross-linking agent includes N-(3-dimethylaminopropyl)-N′-ethylcarbodiimide hydrochloride (EDC•HCl), N-hydroxysuccinimide (NHS) or a combination thereof.

In an embodiment of the invention, a reaction temperature of the cross-linking reaction ranges from about 4° C. to 60° C.

In an embodiment of the invention, a reaction time of the cross-linking reaction ranges from about 4 hours to 24 hours.

In an embodiment of the invention, a surface of the self-assembled nanostructure is further modified with a protein molecule having specificity.

In an embodiment of the invention, the protein molecule includes an epidermal growth factor receptor (EGFR) or CD133 protein.

In an embodiment of the invention, in preparing the amphiphilic chitosan-containing solution, the content of the amphiphilic chitosan is about 0.05 part by weight to 1 part by weight based on 100 parts by weight of the solution.

In an embodiment of the invention, the contrast agent compound includes a magnetic resonance imaging (MRI) contrast agent compound.

In an embodiment of the invention, the MRI contrast agent compound includes gadodiamide, gadopentetate dimeglumine, gadoterate meglumine or a combination thereof.

Based on the above, in the self-assembled nanostructure of the invention, the contrast agent compound is grafted to the amphiphilic chitosan, and due to chemical bonding between these two, the contrast agent compound is prevented from being released into tissue of an organism to cause any harm to the organism. In addition, the bonding between the amphiphilic chitosan and the contrast agent compound has a synergistic effect, which further improves the contrasting ability of the contrast agent compound. In addition, the self-assembled nanostructure of the invention may be modified with a protein molecule having specificity, so that the self-assembled nanostructure of the invention exhibits recognition specificity, and efficiency in drug administration is improved.

To make the above features and advantages of the invention more comprehensible, embodiments accompanied with drawings are explained in detail as follows.

### BRIEF DESCRIPTION OF THE DRAWINGS

FIG. 1 is a schematic diagram illustrating a self-assembled nanostructure according to an embodiment of the invention.

FIG. 2 is a schematic diagram illustrating encapsulating a drug molecule by a self-assembled nanostructure according to an embodiment of the invention.

FIG. 3 is a schematic structural diagram illustrating encapsulating a drug molecule and a fluorescence contrast agent compound by a self-assembled nanostructure according to another embodiment of the invention.

FIG. 4 is a flowchart illustrating a method for preparing a self-assembled nanostructure according to an embodiment of the invention.

FIG. 5 is an infrared spectrum of gadodiamide, carboxymethyl hexanoyl chitosan and carboxymethyl hexanoyl chitosan having gadodiamide grafted thereto according to an embodiment of the invention.

FIG. 6 illustrates a comparison of encapsulation rates of a self-assembled nanostructure for different drug molecules according to an embodiment of the invention.

FIG. 7 illustrates a comparison of cell endocytosis amounts between a self-assembled nanostructure modified with an epidermal growth factor receptor (EGFR) and a self-assembled nanostructure without being modified with an EGFR.

### DETAILED DESCRIPTION OF DISCLOSED EMBODIMENTS

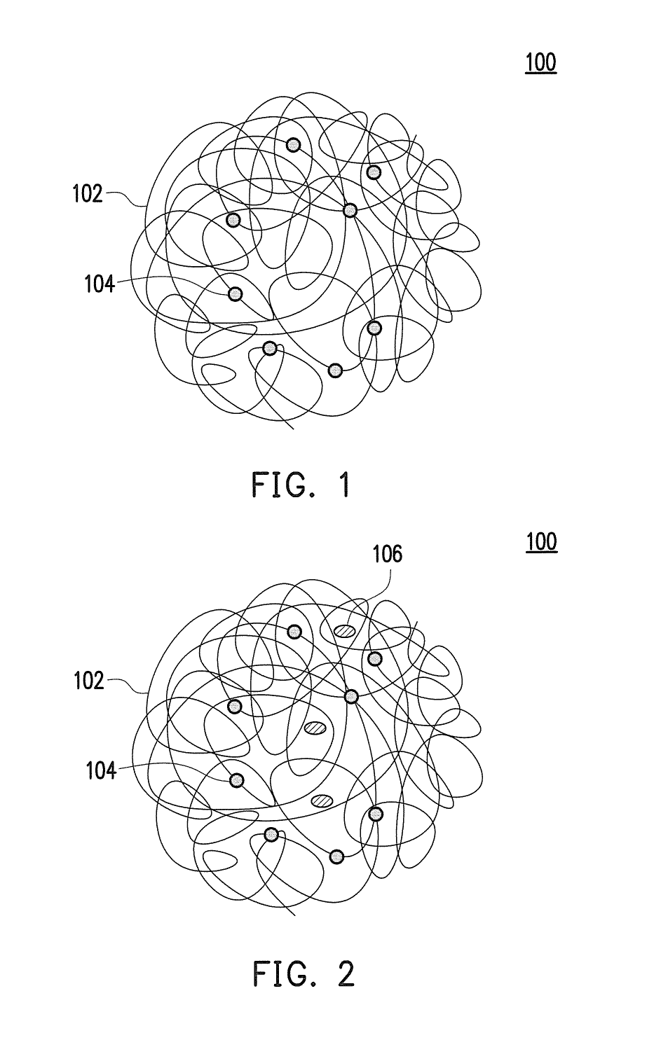

FIG. 1 is a schematic diagram illustrating a self-assembled nanostructure according to an embodiment of the invention.

Referring to FIG. 1, a self-assembled nanostructure 100 of the invention includes an amphiphilic chitosan 102 and a contrast agent compound 104, wherein the contrast agent compound 104 is grafted to the amphiphilic chitosan 102. In other words, there is chemical bonding instead of physical adsorption between the amphiphilic chitosan 102 and the contrast agent compound 104. As shown in FIG. 1, in an embodiment, in an appropriate environment (e.g., an aqueous solution), the amphiphilic chitosan 102 having the contrast agent compound 104 grafted thereto is entangled and self-assembles into a spherical nanoparticle. More specifically, the amphiphilic chitosan 102 of the invention has both hydrophilic and hydrophobic functional groups, and has a self-assembly property in aqueous solution, and is therefore capable of forming the self-assembled nanostructure 100 of the invention by having the contrast agent compound 104 grafted thereto.

In an embodiment, the amphiphilic chitosan 102 of the invention is, for example, carboxymethyl hexanoyl chitosan (CHC), deoxycholic acid modified carboxymethylated chitosan (DCMC), lauroyl sulfated chitosan (LSC) or methylpyrrolidone chitosan (MPC). The amphiphilic chitosan 102 of the invention, by having the contrast agent compound 104 grafted thereto, has a critical micelle concentration of between about 0.010 mg/ml and 0.030 mg/ml, such as about 0.026 mg/ml to 0.028 mg/ml. That is, the amphiphilic chitosan 102 of the invention, by having the contrast agent compound 104 grafted thereto, is capable of self-assembly at extremely low concentration to form the self-assembled nanostructure 100 of the invention.

The contrast agent compound 104 includes a magnetic resonance imaging (MRI) contrast agent compound. In an embodiment, the MRI contrast agent compound is, for example, a gadolinium complex, a manganese complex, a chromium complex or an iron complex. The gadolinium complex is, for example, gadodiamide, gadopentetate dimeglumine, gadoterate meglumine or a combination thereof. In an embodiment, as shown in FIG. 1, the contrast agent compound 104 of the invention is located inside the self-assembled nanostructure 100. Accordingly, the probability of the contrast agent compound 104 contacting an organism is reduced. Therefore, the self-assembled nanostructure 100 of the invention has extremely low toxicity. In FIG. 1, all the contrast agent compounds 104 are located inside the self-assembled nanostructure 100. However, the invention is not limited thereto. In other embodiments (not illustrated), some of the contrast agent compounds 104 may be located outside the self-assembled nanostructure 100.

In an embodiment, the amphiphilic chitosan 102 has a weight average molecular weight of about 1,000 to 60,000, such as about 5,000 to 60,000, about 10,000 to 50,000 or about 20,000 to 40,000. The weight average molecular weight of the amphiphilic chitosan 102 affects the particle size of the self-assembled nanostructure 100. In an embodiment, the self-assembled nanostructure 100 of the invention has a particle size of about 5 nm to 500 nm, such as about 10 nm to 500 nm, about 50 nm to 400 nm or about 100 nm to 300 nm. If the particle size of the self-assembled nanostructure 100 is smaller than about 5 nm, the self-assembled nanostructure 100 is easily carried away by the blood. If the particle size of the self-assembled nanostructure 100 is larger than about 500 nm, the self-assembled nanostructure 100 has difficulty entering tumor cells in the organism.

In an embodiment, based on 100 parts by weight of the self-assembled nanostructure 100, the contrast agent compound 104 is in an amount of about 1 part by weight to 30 parts by weight, such as about 1 part by weight to 20 parts by weight, about 5 parts by weight to 20 parts by weight or about 10 parts by weight to 15 parts by weight. If the contrast agent compound 104 is in an amount of less than about 1 part by weight, the self-assembled nanostructure 100 has insufficient contrasting ability. If the contrast agent compound 104 is in an amount of more than about 30 parts by weight, the self-assembled nanostructure 100 has difficulty self-assembling into a spherical shape in aqueous solution.

The self-assembled nanostructure 100 of the invention can be used to encapsulate a drug molecule, so that the drug molecule is protected by the self-assembled nanostructure 100 during the transportation inside the organism. In addition, by means of the contrasting ability of the self-assembled nanostructure 100 of the invention, the position of the drug molecule encapsulated by the self-assembled nanostructure 100 in the organism is easily observed.

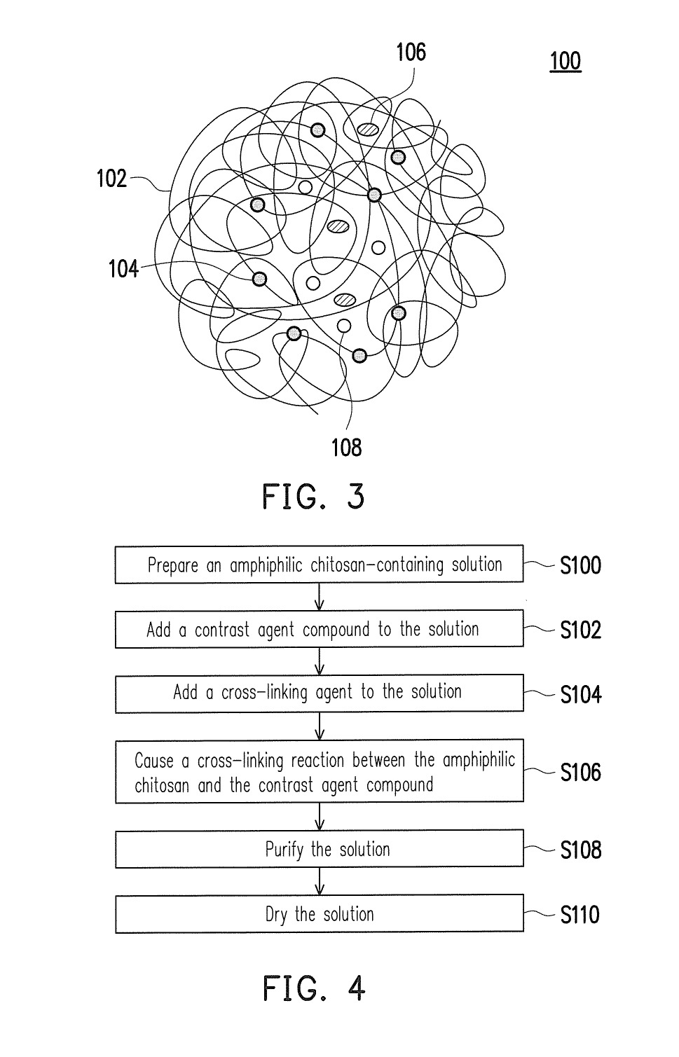

FIG. 2 is a schematic diagram illustrating encapsulating a drug molecule by a self-assembled nanostructure according to an embodiment of the invention. FIG. 3 is a schematic structural diagram illustrating encapsulating a drug molecule and a fluorescence contrast agent compound by a self-assembled nanostructure according to another embodiment of the invention.

Referring to FIG. 2 and FIG. 3, the self-assembled nanostructure 100 encapsulates a drug molecule 106. More specifically, the entangled network structure of the self-assembled nanostructure 100 traps the drug molecule 106 therein. In an embodiment, the drug molecule is, for example, cis-diamminedichloridoplatinum(II) or a derivative thereof, gemcitabine or a derivative thereof, or demethoxycurcumin or a derivative thereof. It is noted that the drug molecule 106 is physically encapsulated in the self-assembled nanostructure 100. That is, there is no chemical bonding between the drug molecule 106 and the self-assembled nanostructure 100. Therefore, the drug molecule 106 is more easily released from the self-assembled nanostructure 100 so as to be absorbed by the organism.

In an embodiment, the self-assembled nanostructure 100 further encapsulates a fluorescence contrast agent compound 108 so as to further improve the contrasting ability, as shown in FIG. 3. More specifically, the entangled network structure of the self-assembled nanostructure 100 traps the fluorescence contrast agent compound 108 therein. In an embodiment, the fluorescence contrast agent compound 108 is, for example, fluorescein isothiocyanate (FITC), C₈₃H₉₅N₁₃O₂₃S₂Zn₂ (e.g., PSVue™794) or C₅₇H₅₈N₁₄O₁₈SZn₂ (e.g., PSVue™480).

It is noted that the fluorescence contrast agent compound 108 is also physically encapsulated in the self-assembled nanostructure 100. That is, there is no chemical bonding between the fluorescence contrast agent compound 108 and the self-assembled nanostructure 100.

In an embodiment, the contrast agent compound of the invention is, for example, an FDA certified metal complex. Therefore, there is an extremely low probability of the self-assembled nanostructure of the invention causing serious allergic reaction in the human body.

FIG. 4 is a flowchart illustrating a method for preparing a self-assembled nanostructure according to an embodiment of the invention.

Referring to FIG. 4, in step S100, an amphiphilic chitosan-containing solution is first prepared. In an embodiment, the amphiphilic chitosan is, for example, carboxymethyl hexanoyl chitosan (CHC), deoxycholic acid modified carboxymethylated chitosan (DCMC), lauroyl sulfated chitosan (LSC) or methylpyrrolidone chitosan (MPC). In an embodiment, in preparing the amphiphilic chitosan-containing solution, based on 100 parts by weight of the solution, the content of the amphiphilic chitosan is about 0.05 part by weight to 1 part by weight, such as about 0.1 part by weight to 1 part by weight, about 0.15 part by weight to 0.85 part by weight or about 0.2 part by weight to 0.7 part by weight.

Next, in step S102, a contrast agent compound is added to the solution. In an embodiment, the contrast agent compound includes a magnetic resonance imaging (MRI) contrast agent compound. The MRI contrast agent compound is, for example, a gadolinium complex, a manganese complex, a chromium complex or an iron complex. The gadolinium complex is, for example, gadodiamide, gadopentetate dimeglumine or gadoterate meglumine.

Then, in step S104, a cross-linking agent is added to the solution. In an embodiment, the cross-linking agent is, for example, N-(3-dimethylaminopropyl)-N′-ethylcarbodiimide hydrochloride, N-hydroxysuccinimide or a combination thereof.

Then, in step S106, a cross-linking reaction is caused between the amphiphilic chitosan and the contrast agent compound. In an embodiment, the reaction temperature of the cross-linking reaction is, for example, about 4° C. to 60° C., such as about 10° C. to 60° C., about 20° C. to 60° C. or about 40° C. to 60° C. The reaction time of the cross-linking reaction is, for example, about 4 hours to 24 hours, such as about 8 hours to 24 hours, about 12 hours to 24 hours or about 18 hours to 24 hours. In the invention, by controlling parameters such as the amount of the cross-linking agent, the reaction temperature and the reaction time of the cross-linking reaction, self-assembled nanostructures having different contents of contrast agents are prepared.

Subsequently, in step S108, the solution is purified. In an embodiment, the purification method is, for example, purification by dialysis. The solution used for dialysis is, for example, water or ethanol.

Next, in step S110, the solution is dried to obtain yellow powder. In an embodiment, the drying method is, for example, drying by baking or freeze-drying. Then, the yellow powder is dissolved in an aqueous solution to obtain the self-assembled nanostructure 100 of the invention.

In an embodiment, the surface of the self-assembled nanostructure 100 is further modified with a protein molecule having specificity. The protein molecule is, for example, an epidermal growth factor receptor (EGFR) or CD133 protein. After the surface of the self-assembled nanostructure 100 of the invention is modified with the protein molecule having specificity, the self-assembled nanostructure 100 exhibits recognition specificity for different targets. For example, the protein molecule having specificity is an antibody and is used to modify the surface of the self-assembled nanostructure 100 of the invention. In this case, the self-assembled nanostructure 100 of the invention has specificity for a corresponding antigen.

The self-assembled nanostructure of the invention is prepared by a simple process, which reduces the difficulty of mass production. Most of the solvents used in the production process of the self-assembled nanostructure of the invention are solvents having lower toxicity, such as water, methanol or isopropyl alcohol, etc., which increases safety of the self-assembled nanostructure.

### EMBODIMENTS

The self-assembled nanostructure of the invention is prepared, for example, in the following manner. 0.5 g of carboxymethyl hexanoyl chitosan, 0.2 g of gadodiamide and 0.65 g of N-(3-dimethylaminopropyl)-N′-ethylcarbodiimide hydrochloride were prepared and dissolved in 100 ml of phosphate buffer. The phosphate buffer had a pH of 7.4. After the resultant was stirred at a low temperature (4° C.) for 30 minutes, 0.39 g of N-hydroxysuccinimide was added and the resultant was stirred at a low temperature (4° C.) for 24 hours. After the reaction was completed, the solution was subjected to dialysis with pure water for 48 hours and then with ethanol for 24 hours. Next, the solution was dried by baking and then crushed by stirring, so as to obtain yellow powder. The yellow powder was dissolved in an aqueous solution to obtain the self-assembled nanostructure of the invention.

In an embodiment, as shown in Table 1 below, by adjusting the reaction time of the cross-linking reaction, self-assembled nanostructures having different contents of contrast agents were obtained.

TABLE 1

| | | Content of | Content of |
|---|---|---|---|
| | | Gadodiamide | Gadolinium |
| | | in Self- | in Self- |
| | | assembled | assembled |
| | | Nano- | Nano- |
| Reaction | | structure | structure |
| Time | Gadodiamide:EDC•HCl:NHS | (wt %) | (wt %) |
| 4 hours | 1:2:2 | 15.86 wt % | 4.77 wt % |
| 8 hours | 1:2:2 | 12.39 wt % | 3.62 wt % |
| 12 hours | 1:2:2 | 13.36 wt % | 3.94 wt % |
| 18 hours | 1:2:2 | 9.92 wt % | 2.84 wt % |
| 24 hours | 1:2:2 | 17.10 wt % | 5.20 wt % |

In an embodiment, as shown in Table 2 below, by adjusting the reaction temperature of the cross-linking reaction, self-assembled nanostructures having different contents of contrast agents were obtained.

TABLE 2

| | | Content of | Content of |
|---|---|---|---|
| | | Gadodiamide | Gadolinium |
| | | in Self-assembled | in Self-assembled |
| Reaction | Reaction | Nanostructure | Nanostructure |
| Time | Temperature | (wt %) | (wt %) |
| 4 hours | 4° C. | 15.98 wt % | 4.81 wt % |
| 4 hours | Room temperature | 15.86 wt % | 4.77 wt % |
| 4 hours | 40° C. | 13.90 wt % | 4.12 wt % |
| 4 hours | 50° C. | 11.57 wt % | 3.36 wt % |
| 4 hours | 60° C. | 9.03 wt % | 2.57 wt % |

In an embodiment, as shown in Table 3 below, by adjusting the proportion of the cross-linking agent, self-assembled nanostructures having different contents of contrast agents were obtained.

TABLE 3

| | | Content of | Content of |
|---|---|---|---|
| | | Gadodiamide | Gadolinium |
| | | in Self- | in Self- |
| | | assembled | assembled |
| | | Nano- | Nano- |
| Reaction | | structure | structure |
| Time | Gadodiamide:EDC•HCl:NHS | (wt %) | (wt %) |
| 4 hours | 1:2:5 | 14.57 wt % | 4.34 wt % |
| 4 hours | 1:2:1 | 10.54 wt % | 3.04 wt % |
| 4 hours | 1:5:2 | 15.94 wt % | 4.80 wt % |
| 4 hours | 1:4:4 | 12.57 wt % | 3.68 wt % |

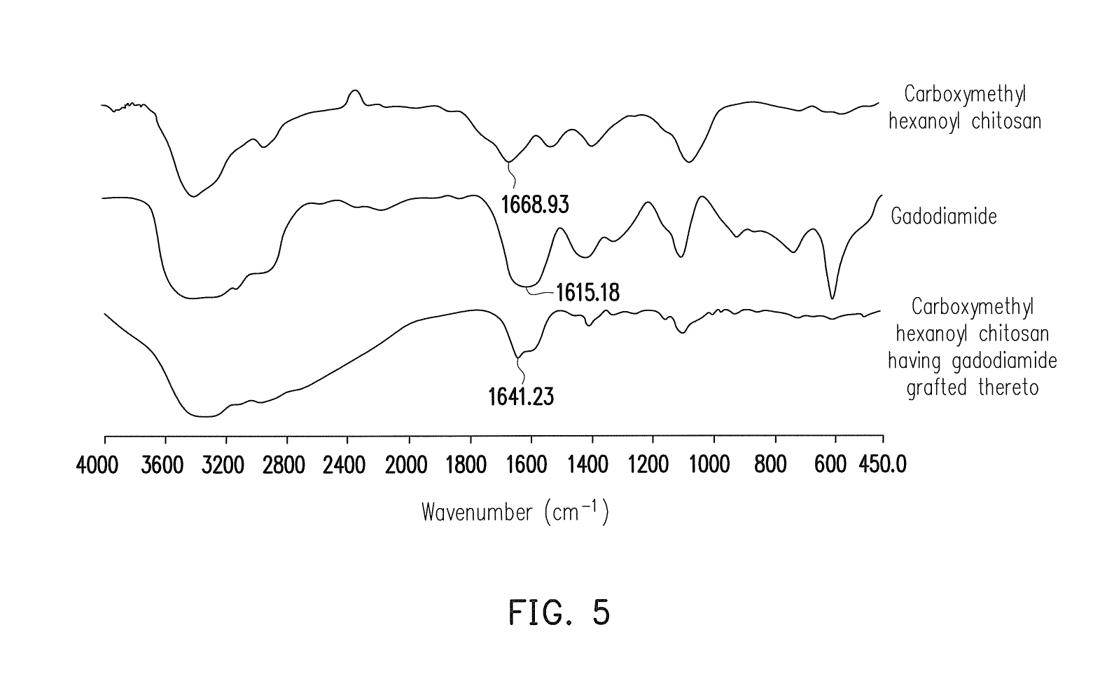

FIG. 5 is an infrared spectrum of gadodiamide, carboxymethyl hexanoyl chitosan and carboxymethyl hexanoyl chitosan having gadodiamide grafted thereto according to an embodiment of the invention.

Referring to FIG. 5, the C═O signal of carboxymethyl hexanoyl chitosan appears at 1668.93 cm⁻¹, and the C═O signal of carboxymethyl hexanoyl chitosan having gadodiamide grafted thereto appears at 1641.23 cm⁻¹. More specifically, the C═O signal is shifted after the gadodiamide is grafted to the carboxymethyl hexanoyl chitosan. The carboxymethyl hexanoyl chitosan with gadodiamide grafted thereto has a tertiary amine, while the carboxymethyl hexanoyl chitosan without gadodiamide grafted thereto only has a secondary amine. The electron-donating behavior of the alkyl group on the tertiary amine reduces the bond energy of C═O. Hence the C═O signal of the carboxymethyl hexanoyl chitosan with gadodiamide grafted thereto has a smaller wavenumber. It is thus proved that in the self-assembled nanostructure of the invention, there is chemical bonding between carboxymethyl hexanoyl chitosan and gadodiamide.

Relaxivities of a self-assembled nanostructure of the invention, gadodiamide, and carboxymethyl hexanoyl chitosan that physically encapsulates gadodiamide are as shown in Table 4. Hereinafter, the symbol “CHC+Gd” represents that there is only physical mixing between gadodiamide and carboxymethyl hexanoyl chitosan, while the symbol “CHC−Gd” represents that chemical bonding exists between gadodiamide and carboxymethyl hexanoyl chitosan (i.e., the self-assembled nanostructure of the invention).

TABLE 4

| | Relaxivity (mM⁻¹s⁻¹) |
|---|---|
| CHC − Gd (Self-assembled | 4.2729 |
| Nanostructure of | |
| the Invention) | |
| CHC + Gd | 1.2277 |
| Gadodiamide | 1.8158 |

Referring to Table 4, it is proved that the relaxivity of the self-assembled nanostructure of the invention is higher than the relaxivities of the single gadodiamide and CHC+Gd. As a molecule grafted to a paramagnetic metal of a contrast agent is larger, a rotation time between the paramagnetic metal and a solvent molecule becomes longer, so that the relaxivity of the contrast agent is enhanced. The higher the relaxivity of the contrast agent, the better the contrast effect, i.e., the more excellent the contrasting ability. In the self-assembled nanostructure of the invention, gadolinium (paramagnetic metal) has an amphiphilic chitosan (polymer) grafted thereto, and thus has a higher relaxivity than the relaxivities of the single gadodiamide and CHC+Gd. Therefore, the self-assembled nanostructure of the invention exhibits a more excellent contrast effect than that of the single gadodiamide or CHC+Gd. In other words, the contrast agent compound of the invention, after being grafted to the amphiphilic chitosan, produces a synergistic effect on the contrasting ability of the contrast agent compound. Moreover, this synergistic effect only occurs when chemical bonding is present between gadodiamide and carboxymethyl hexanoyl chitosan (i.e., the self-assembled nanostructure of the invention), and does not occur when there is only physical mixing between these two.

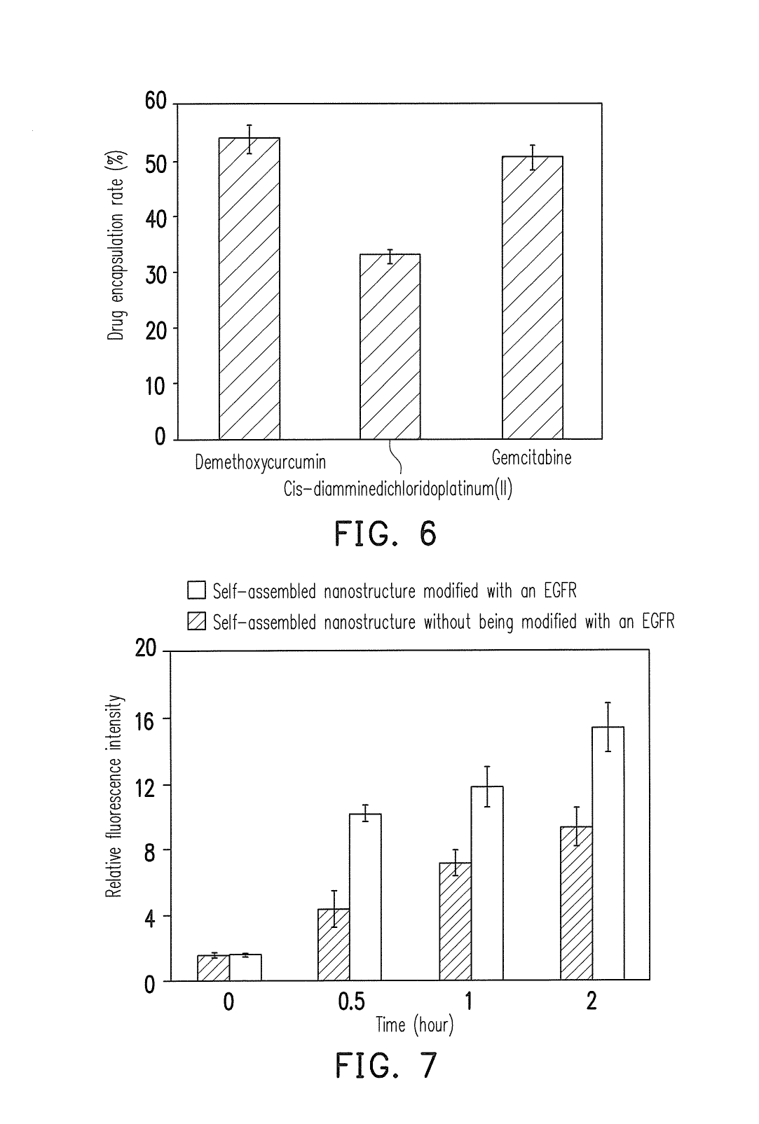

FIG. 6 illustrates a comparison of encapsulation rates of a self-assembled nanostructure for different drug molecules according to an embodiment of the invention.

The self-assembled nanostructure of the invention, after encapsulating a drug molecule in an aqueous solution for 12 hours, exhibits an excellent encapsulation rate for the drug molecule. As shown in FIG. 6, the self-assembled nanostructure of the invention, after encapsulating a drug molecule such as demethoxycurcumin, cis-diamminedichloridoplatinum (II) or gemcitabine, etc. in an aqueous solution for 12 hours, exhibits an excellent encapsulation rate for the aforementioned drug molecules. Therefore, the self-assembled nanostructure of the invention is valuable in application in the biomedical field.

FIG. 7 illustrates a comparison of cell endocytosis amounts between a self-assembled nanostructure modified with an epidermal growth factor receptor (EGFR) and a self-assembled nanostructure without being modified with an EGFR.

In an embodiment of the invention, the self-assembled nanostructure modified with an EGFR and the self-assembled nanostructure without being modified with an EGFR each encapsulate a fluorescence contrast agent compound (e.g., PSVue™794 or PSVue™480), and are then respectively placed in lung cancer cell lines (A549) for cell culture for 4 hours. In the invention, a self-assembled nanostructure encapsulating a fluorescence contrast agent compound is obtained, for example, in the following manner. Carboxymethyl hexanoyl chitosan having gadodiamide grafted thereto was dissolved in water. The concentration of the carboxymethyl hexanoyl chitosan having gadodiamide grafted thereto was 0.05 wt %. 500 g/ml of a fluorescence contrast agent compound (e.g., PSVue™794 or PSVue™480) was added to the solution and stirred for 12 hours. The solution was then subjected to dialysis with pure water to obtain the self-assembled nanostructure encapsulating the fluorescence contrast agent compound.

By comparing relative fluorescence intensities of the lung cancer cell lines (A549), cell endocytosis amounts of the lung cancer cell lines (A549) for the self-assembled nanostructure modified with an EGFR and the self-assembled nanostructure without being modified with an EGFR are obtained. As shown in FIG. 7, the lung cancer cell line (A549) has an excellent cell endocytosis amount for the self-assembled nanostructure modified with an EGFR. Therefore, the self-assembled nanostructure of the invention is valuable in application in the biomedical field.

In summary, in the self-assembled nanostructure of the invention, the contrast agent compound is grafted to the amphiphilic chitosan, and due to chemical bonding between these two, the contrast agent compound is prevented from being released into tissue of an organism to cause any harm to the organism. In addition, the bonding between the amphiphilic chitosan and the contrast agent compound has a synergistic effect, which further improves the contrasting ability of the contrast agent compound. In addition, the self-assembled nanostructure of the invention may be modified with a protein molecule having specificity, so that the self-assembled nanostructure of the invention exhibits recognition specificity, and the efficiency in drug administration is improved.

Although the invention has been disclosed with reference to the above embodiments, it will be apparent to persons of ordinary skill in the art that modifications to the described embodiments may be made without departing from the spirit of the invention. Accordingly, the scope of the invention will be defined by the attached claims and not by the above detailed descriptions.

1. A self-assembled nanostructure, comprising:
an amphiphilic chitosan; and
a contrast agent compound grafted to the amphiphilic chitosan.

2. The self-assembled nanostructure of claim 1, wherein the amphiphilic chitosan comprises carboxymethyl hexanoyl chitosan (CHC), deoxycholic acid modified carboxymethylated chitosan (DCMC), lauroyl sulfated chitosan (LSC) or methylpyrrolidone chitosan (MPC).

3. The self-assembled nanostructure of claim 1, wherein the contrast agent compound comprises a magnetic resonance imaging (MRI) contrast agent compound.

4. The self-assembled nanostructure of claim 3, wherein the MRI contrast agent compound comprises gadodiamide, gadopentetate dimeglumine, gadoterate meglumine or a combination thereof.

5. The self-assembled nanostructure of claim 1, wherein the amphiphilic chitosan has a weight average molecular weight of about 1,000 to 60,000.

6. The self-assembled nanostructure of claim 1, wherein the self-assembled nanostructure is entangled to form a spherical nanoparticle having a particle size of about 5 nm to 500 nm.

7. The self-assembled nanostructure of claim 1, wherein the contrast agent compound is in an amount of about 1 part by weight to 30 parts by weight based on 100 parts by weight of the self-assembled nanostructure.

8. The self-assembled nanostructure of claim 1, wherein the self-assembled nanostructure encapsulates a fluorescence contrast agent compound.

9. The self-assembled nanostructure of claim 8, wherein the fluorescence contrast agent compound comprises fluorescein isothiocyanate (FITC), C₈₃H₉₅N₁₃O₂₃S₂Zn₂ or C₅₇H₅₈N₁₄O₁₈SZn₂.

10. The self-assembled nanostructure of claim 1, wherein the self-assembled nanostructure encapsulates a drug molecule.

11. The self-assembled nanostructure of claim 10, wherein the drug molecule comprises cis-diamminedichloridoplatinum(II) or a derivative thereof, gemcitabine or a derivative thereof, or demethoxycurcumin or a derivative thereof.

12. A method for preparing a self-assembled nanostructure, comprising:
preparing an amphiphilic chitosan-containing solution;
adding a contrast agent compound to the solution;
adding a cross-linking agent to the solution;
causing a cross-linking reaction between the amphiphilic chitosan and the contrast agent compound;
purifying the solution; and
drying the solution.

13. The method for preparing a self-assembled nanostructure of claim 12, wherein the cross-linking agent comprises N-(3-dimethylaminopropyl)-N′-ethylcarbodiimide hydrochloride (EDC •HCl), N-hydroxysuccinimide (NHS) or a combination thereof.

14. The method for preparing a self-assembled nanostructure of claim 12, wherein a reaction temperature of the cross-linking reaction ranges from about 4° C. to 60° C.

15. The method for preparing a self-assembled nanostructure of claim 12, wherein a reaction time of the cross-linking reaction ranges from about 4 hours to 24 hours.

16. The method for preparing a self-assembled nanostructure of claim 12, further comprising modifying a surface of the self-assembled nanostructure with a protein molecule having specificity.

17. The method for preparing a self-assembled nanostructure of claim 16, wherein the protein molecule comprises an epidermal growth factor receptor (EGFR) or CD133 protein.

18. The method for preparing a self-assembled nanostructure of claim 12, wherein in preparing the amphiphilic chitosan-containing solution, a content of the amphiphilic chitosan is about 0.05 part by weight to 1 part by weight based on 100 parts by weight of the solution.

19. The method for preparing a self-assembled nanostructure of claim 12, wherein the contrast agent compound comprises a magnetic resonance imaging (MRI) contrast agent compound.

20. The method for preparing a self-assembled nanostructure of claim 19, wherein the MRI contrast agent compound comprises gadodiamide, gadopentetate dimeglumine, gadoterate meglumine or a combination thereof.
